(19) United States
(12) Patent Application Publication
(10) Pub. No.: US 2005/0175619 A1
(43) Pub. Date: Aug. 11, 2005

(54) Methods of producing antibody conjugates

(76) Inventors: Robert Duffy, Castro Valley, CA (US); Rick Powers, Palo Alto, CA (US)

Correspondence Address: HOWREY SIMON ARNOLD & WHITE, LLP, c/o IP DOCKETING DEPARTMENT, 2941 FAIRVIEW PARK DRIVE, SUITE 200, FALLS CHURCH, VA, 22042-2924, US

(21) Appl. No.: 11/050,981

(22) Filed: Feb. 4, 2005

Related U.S. Application Data
(60) Provisional application No. 60/542,787, filed on Feb. 5, 2004.

(51) Int. Cl. A61K 39/395; C07K 16/46

(52) U.S. Cl. 424/178.1; 530/391.1

(57) ABSTRACT

The present invention is directed to improved processes for making antibody conjugates with effector moieties, wherein the conjugation reaction is carried out in a solution comprising an alcohol. The alcohol solvated conjugation reaction may be conducted at room temperature and/or in a single-pot enclosed system that minimizes the safety risks of conjugating highly cytotoxic agents. The disclosed conjugation process is capable of producing high monomer content, commercial scale quantities of antibody conjugates in a single batch.

### CROSS REFERENCE TO RELATED APPLICATIONS

The present application claims priority of U.S. provisional patent application 60/542,787, filed Feb. 5, 2004, which is hereby incorporated by reference herein.

### FIELD OF THE INVENTION

The present invention relates to the field of immunology and biochemistry. In particular, it concerns improved methods of making antibody conjugates to effector moieties, wherein the conjugates are useful in the diagnosis and treatment of disease.

### BACKGROUND OF THE INVENTION

Antibody-based drugs are widely used in clinical treatment of disease, most notably cancer and autoimmune diseases. As one type of antibody-based therapeutics, antibody conjugates, which comprise a targeting antibody linked to an effector moiety, have shown great clinical potential in diagnosis and treatment of disease.

Methods for making antibody conjugates have been described in the art, including Francisco, J. A., et al., Blood 102: 1458-1465 (2003); Doronina, et al., Nature Biotechnology 21: 778-784 (2003); U.S. Pat. Nos. 5,635,483 and 6,214,345; U.S. patent application Publication Ser. Nos. 20030083263 and 20050009751 A1; and PCT Publication No. WO2002088172; each of which is hereby incorporated by reference herein in its entirety. These existing methods for making antibody conjugates all use aprotic organic solvents such as dimethylsulfoxide (DMSO) or acetonitrile (ACN) as solubilizing agents during the conjugation reaction. In addition, the methods often give rise to excessive undesirable aggregation of clusters comprising multiple antibodies and agents. In the art, this aggregation is considered as the most common cause of failures during the batch production of antibody conjugates. Furthermore, the existing methods for preparing antibody conjugates require multiple material transfers during the production process. Consequently, these prior art processes create a substantial risk of exposure to the cytotoxic agents that are being conjugated to the antibody. Among the transfer steps, the existing methods require the use of sizing (i.e. “filtration” or “desalting”) columns, e.g., to remove excess dithiothreitol (DTT) used in the initial antibody reduction step, and then to remove the residual free cytotoxic agent after the conjugation reaction. These sizing columns have relatively low limits on liquid volume throughput, and consequently are not reasonably scaled to the large volumes necessary for clinical and commercial production.

In view of the above described deficiencies in the existing methods, it is of importance to seek safer and more efficient ways of producing antibody conjugates, especially for large scale commercial production.

### SUMMARY OF THE INVENTION

The present invention includes a process of producing a conjugate of an antibody and at least one effector moiety, said process comprising: performing a reaction coupling the antibody to the effector moiety in a solution comprising at least 5% by volume of an alcohol. In one embodiment the process is carried out wherein the alcohol is selected from the group consisting of ethanol and isopropyl alcohol. In other embodiments, the process may be carried out wherein the coupling reaction solution comprises an alcohol at a concentration of about 5% to 50% by volume.

The presence of the alcohol in the coupling reaction facilitates the use of much higher antibody concentrations with minimal, if any, aggregation that typically occurs at these concentrations. In preferred embodiments the above process of producing a conjugate of an antibody and at least one effector moiety is carried wherein said antibody in the solution has a concentration of between about 10 mg/mL and 50 mg/mL, with exemplary concentrations including all integer values between and including 10 and 50. In a more preferred embodiment, the antibody concentration is in the range of about 10 mg/mL to 30 mg/mL, with exemplary concentrations including all integer values between and including 10 and 30, for example the concentrations of about 10, 12, 15, 16, 17, 20, 23, 25, 28 or 30 mg/mL.

In addition, the presence of the alcohol in the antibody effector moiety coupling reaction facilitates a process that may be conveniently scaled up for production of large quantities of antibody conjugate (e.g. up to, and including about 5000 mg of antibody conjugate in a single batch). In one embodiment of the process, the amount of antibody conjugate produced is between about 100 mg and 400 mg.

Despite the high antibody concentrations used, the process of the present invention results in low antibody aggregation, and accordingly much higher levels of monomer antibody-conjugate formation (e.g. at least 75%, 80%, 85%, 90%, 95%, 95%, 99% and even higher percentages of monomer antibody conjugate). In addition, the process of the present invention results in high ratios of effector moieties coupled per antibody molecule (e.g. in a range of 6-10 moieties per antibody).

In another embodiment, the present invention includes a process of producing a conjugate of an antibody and at least one effector moiety, wherein the coupling reaction is performed above 4° C., and preferably at between about 15° C. and 25° C., or at room temperature (i.e. about 22° C.).

The present invention may be used with any antibody (or immunoglobulin) molecule. Thus, the present invention also includes a process of producing a conjugate of an antibody and at least one effector moiety, wherein the antibody conjugate comprises a monoclonal antibody, and/or wherein said antibody conjugate comprises a chimeric, humanized, or fully human antibody. Alternatively, the process may be carried out wherein the antibody is an antigen binding fragments selected from the list consisting of Fab′, F(ab′)₂, Fab, Fv, rIgG, and scFv fragments.

Similarly, the present process of producing a conjugate of an antibody and at least one effector moiety may be carried out with a range of effector moieties including those selected from the group consisting of a therapeutic moiety and a reporting moiety. In preferred embodiment, the process may be carried wherein the therapeutic moiety is selected from the group consisting of a cytotoxic agent, a chemotherapeutic agent, an immunotoxin, and a drug. In one preferred embodiment, the effector moiety is a cytotoxic agent or a chemotherapeutic agent. In another preferred embodiment, the effector moiety is a cytotoxic agent selected from the group consisting of diphtheria A chain, exotoxin A chain, ricin A chain, abrin A chain, calicheamicin, curcin, crotin, phenomycin, enomycin, dolastatin 10, auristatin E, auristatin F, and MMAE. Preferred chemotherapeutic agents may be selected from the group consisting of adriamycin, doxorubicin, doxil, epirubicin, 5-fluorouracil, cytosine arabinoside, cyclophosphamide, thiotepa, busulfan, cytoxin, taxoids, paclitaxel, doxetaxel, toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, 5-FU, 6-thioguanine, 6-mercaptopurine, actinomycin D, VP-16, chlorambucil, melphalan, tamoxifen, and onapristone

In another embodiment, the present invention includes a method of producing an antibody conjugate with at least one effector moiety, said method comprising:
a. contacting an antibody with a reducing agent;
b. performing a reaction coupling the reduced antibody to an effector moiety in a solution comprising at least 5% by volume of an alcohol; and
c. separating the antibody conjugate from uncoupled effector moiety.

In preferred embodiments, the above method may be carried out wherein said reducing agent is dithiothreitol, and/or wherein the residue of said reducing agent is removed by ultrafiltration between step a and step b. In another preferred embodiment, the method may be carried out wherein said uncoupled effector moiety is separated from the antibody conjugate by diafiltration.

Significantly, the present invention includes a method of producing an antibody conjugate with at least one effector moiety, wherein each step of said method is performed within an ultrafiltration apparatus (e.g. an ultrafiltration skid). In one preferred embodiment of this method, all three steps of the method are performed in a single recirculating system comprising a recirculation pump connected to a tangential flow filtration (TFF) device. In a most preferred embodiment, all three steps of the method are performed in a single-pot system comprising a single solution of antibody, reduced antibody, and/or antibody conjugate that remains in the system until all three steps are complete.

### BRIEF DESCRIPTION OF THE DRAWINGS

FIG. 1 depicts a recirculating system useful for making antibody conjugates according to one embodiment of the present invention, wherein the system comprises a recirculation pump (101), a sample reservoir (103), a TFF device (102) comprising a Millipore Pellicon XL ultrafiltration skid, and a waste reservoir (104).

### DETAILED DESCRIPTION OF THE INVENTION

I. Introduction

The present invention discloses a method for producing antibody conjugates that uses alcohol rather than, for example, DMSO or acetonitrile as the organic solvent during the conjugation reaction. The use of alcohol gives rise to a surprising number of benefits including a safer, easily scalable, one-pot process for the clinical and/or commercial production of an antibody conjugates. Among the major advantages of the process for making antibody conjugates disclosed herein relative to the prior art methods are the following:
1) reduced formation of undesirable antibody and antibody conjugate aggregate;
2) increased antibody concentration (10-50 mg/ml) achievable during the conjugation process (due to the reduced aggregation), thereby reducing liquid volumes necessary for large scale production;
3) conjugation process is performed at ambient room temperature (20-25° C.) rather than 4° C., thereby obviating need for refrigeration system for a large-scale reaction vessel;
4) process easily scaled up to commercial levels of antibody conjugate production (e.g. 400 mg to 5000 mg in a single batch) without significant alteration of process;
5) complete antibody conjugation process (including antibody reduction step, and final filtration and formulation steps of conjugate product) may be performed in a “single-pot” enclosed recirculation/ultrafiltration apparatus;
6) “single pot” recirculation process greatly reduces handling and exposure (e.g. through elimination of separate filtration column steps) of reaction solution, thereby minimizing potential safety risks involved in use of highly toxic effector moieties (e.g. vcMMAE).

As a result of the advantages described above, the methods of the present invention are particularly suitable for the safe, large-scale production of antibody conjugates.

II. Antibody Conjugates to Effector Moieties and Their Uses

The present invention is directed to improved methods and processes for making antibody conjugates with effector moieties. The art includes a wide range of antibody conjugates and a corresponding range of uses for them as described in greater detail below. Although the present invention includes a particularly preferred embodiment, the conjugation of the pentapeptide toxin, maleimide-mono-methyl-valine-citrulline-auristatin-E (vcMMAE) to a prostate cancer targeting monoclonal antibody, Pr1, the method disclosed herein is not so limited. As described herein, the surprising advantages yielded by the methods of the present invention arise largely from the improved solubility and reactivity of antibodies in an alcohol solvent during the effector moiety conjugation reaction. Due to the common overall protein structure of immunoglobulins, and the common coupling chemistries used to conjugate to antibodies, one of ordinary skill in the art may immediately recognize that the improvements derived from the alcohol-solvated conjugation process disclosed herein may be applied to a wide range of antibody conjugate systems with little if any routine experimentation.

A. Suitable Antibodies for Conjugation

A variety of antibodies may be conjugated using the methods of the present invention. As used herein, the term “antibody” refers to an immunoglobulin molecule that specifically binds to, or is immunologically reactive with a particular antigen, and includes polyclonal, monoclonal, genetically engineered and otherwise modified forms of antibodies, including but not limited to chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′)₂, Fab, Fv, rIgG, and scFv fragments. The term “scFv” refers to a single chain Fv antibody in which the variable domains of the heavy chain and the light chain from a traditional antibody have been joined via a linker to form one chain. In addition, the term “antibody,” as used in the context of the invention disclosed herein encompasses mixtures of more than one antibody reactive with a specific antigen (e.g., a cocktail of different types of monoclonal antibodies reactive with different epitopes of the TMEFF2 antigen).

Natural antibodies of all species of origins may be conjugated using the methods of the present invention. Natural antibodies are antibodies produced by a host animal after being immunized with an antigen, such as a polypeptide, preferably a human polypeptide. Non-limiting exemplary natural antibodies include antibodies derived from human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety).

The antibodies may also be monoclonal antibodies produced by conventional hybridoma methodology well known in the art, as described originally by Kohler and Milstein, Nature 256: 495-7 (1975); Eur. J. Immunol. 6: 511 (1976). Alternatively, polyclonal antibodies may also be used in the present invention.

Recombinant antibodies may also be conjugated according to the methods of the present invention. Generally, recombinant antibodies can be made in any expression system including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety).

Chimeric antibodies may also be conjugated according to the methods of the present invention. A chimeric antibody is an antibody in which the constant region comes from an antibody of one species (typically human) and the variable region comes from an antibody of another species (typically rodent). Methods for producing chimeric antibodies are well known in the art. See e.g., Morrison, Science 229:1202-1207 (1985); Oi et al., BioTechniques 4:214-221 (1986); Gillies et al., J. Immunol. Methods 125:191-202 (1989); U.S. Pat. Nos. 5,807,715; 4,816,567; and 4,816,397, which are incorporated herein by reference in their entireties.

Humanized antibodies may also be conjugated according to the methods of the present invention. The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85-90%, and preferably at least 95% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos.: 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489-498 (1991); Studnicka et al., Prot. Eng. 7:805-814 (1994); Roguska et al., Proc. Natl. Acad. Sci. 91:969-973 (1994), and chain shuffling (U.S. Pat. No. 5,565,332), all of which are hereby incorporated by reference in their entireties.

Fully human antibodies may also be conjugated according to the methods of the present invention. Fully human antibodies are produced by a variety of techniques, including trioma methodology (Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety)), and non-human transgenic animal approach (Lonberg et al., WO93/12227; U.S. Pat. No. 5,545,806; and Kucherlapati, et al., WO91/10741; U.S. Pat. No. 6,150,584 (each of which is incorporated by reference in its entirety)). Various recombinant antibody library technologies may also be utilized to produce fully human antibodies (See e.g., protocol outlined by Huse et al., Science 246:1275-1281 (1989) and phage-display technology of Dower et al., WO 91/17271 and McCafferty et al., WO 92/01047; U.S. Pat. No. 5,969,108; each of which is incorporated by reference in its entirety).

Fragments of the antibodies, which retain the binding specificity to the desired antigens, are also used in the conjugates in the present invention. In a preferred embodiment of the invention, the antibody fragments are truncated chains (truncated at the carboxyl end). Preferably, these truncated chains possess one or more immunoglobulin activities (e.g., complement fixation activity). Examples of truncated chains include, but are not limited to, Fab fragments (consisting of the VL, VH, CL and CH1 domains); Fd fragments (consisting of the VH and CH1 domains); Fv fragments (consisting of VL and VH domains of a single chain of an antibody); dab fragments (consisting of a VH domain); isolated CDR regions; (Fab′)₂ fragments, bivalent fragments (comprising two Fab fragments linked by a disulfide bridge at the hinge region). The truncated chains can be produced by conventional biochemistry techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CH1 to produce Fab fragments or after the hinge region to produce (Fab′)₂ fragments. Single chain antibodies may be produced by joining V_{L} and V_{H}-coding regions with a DNA that encodes a peptide linker connecting the VL and VH protein fragments.

In particularly preferred embodiments, the method of producing an antibody conjugate may be employed with antibodies that have demonstrated in vivo therapeutic and/or prophylactic uses. Examples of therapeutic and prophylactic antibodies which may be so modified include, but are not limited to: HuZAF™ (fontolizumab) (Protein Design Labs, CA) which is a humanized monoclonal antibody that binds to interferon-γ that is useful for the treatment of severe Crohn's disease; NUVION® (visilizumab) which is a humanized non-FcR binding monoclonal antibody directed at the CD3 antigen on activated T cells useful for the treatment of severe ulcerative colitis (Protein Design Labs, CA); ZENAPAX® (daclizumab) (Protein Design Labs, CA and Roche Pharmaceuticals, Switzerland) which is an immunosuppressive, humanized anti-CD25 useful for the prevention of acute renal allograft rejection, and treatment of asthma symptoms; M200 (Protein Design Labs, CA) which is a chimeric antibody that is a high-affinity functional inhibitor of α5β1 integrin, a cell adhesion molecule that is upregulated on activated endothelial cells, and is useful for the treatment of refractory solid tumors in various cancers; F200 (Protein Design Labs, CA) which is a Fab fragment of the chimeric antibody M200 which is useful for treatment of the wet form of age-related macular degeneration the wet form of age-related macular degeneration; SYNAGIS® (palivizumab) (Medimmune, MD) which is a humanized anti-respiratory syncytial virus (RSV) monoclonal antibody for the treatment of patients with RSV infection; HERCEPTIN® (trastuzumab) (Genentech, CA) which is a humanized anti-HER2 monoclonal antibody for the treatment of patients with metastatic breast cancer; REMICADE® (infliximab) (Centocor, PA) which is a chimeric anti-TNF-α monoclonal antibody for the treatment of patients with Crohn's disease; REOPRO® (abciximab) (Centocor) which is an anti-glycoprotein Ilb/IIIa receptor on the platelets for the prevention of clot formation; XOLAIR® (omalizumab) (Genentech, CA) a humanized anti-IgE for the treatment of asthma symptoms; AVASTIN™ (bevacizumab) (Genentech) which is a humanized anti-VEGF IgG1 antibody useful for treating colorectal cancer; RAPTIVA® (efalizumab) (Genentech, CA) is a humanized anti-CD11a useful for treating psoriasis; LUCENTIS™ (Ranibizumab) (Genentech, CA) which is an antibody fragment to VEGF that is useful for the potential treatment of the wet form of age-related macular degeneration; RITUXAN® (rituxumab) (Genentech, CA) which is a chimeric anti-CD20 IgG1 useful for the treatment of non-Hodgkins lymphoma. MYLOTARG® (gemtuzumab ozogamicin for injection) (Wyeth Pharmaceuticals, PA) which is a recombinant humanized anti-CD33, IgG4 antibody conjugated to the cytotoxic agent, calicheamicin, useful for treating acute myeloid leukemia; and CDP 870 (Celltech, UK) is a PEGylated humanized antibody fragment that binds with high affinity to TNF-α useful for treatment of rheumatoid arthritis.

Other examples are a humanized anti-CD18 F(ab′)2 (Genentech, CA); CDP860 (Celltech, UK) which is a humanized anti-CD18 F (ab′)2; PR0542 (Progenics/Genzyme Transgenics) which is an anti-HIV gp120 antibody fused with CD4; OSTAVIR™ (Protein Design Labs, CA and Novartis) which is a human anti Hepatitis B virus antibody; PROTOVIR™ (Protein Design Labs, CA and Novartis) which is a humanized anti-CMV IgG1 antibody; IC14 (ICOS, WA) which is an anti-CD14 antibody; HUMIRA™ (Cambridge Antibody Technology/BASF) which is a human anti-TNF-α antibody; ERBITUX™ (ImClone Systems) which is a chimeric anti-EGFR IgG antibody; VITAXIN™ (Applied Molecular Evolution/Medimmune) which is a humanized anti-αVβ3 integrin antibody; Campath-1H/LDP-03 (Leukosite) which is a humanized anti CD52 IgG1 antibody; ZAMYL™ (Protein Design Labs, CA and Kanebo) which is a humanized anti-CD33 IgG antibody; LYMPHOCIDE™ (Immunomedics) which is a humanized anti-CD22 IgG antibody; REMITOGEN™ (Protein Design Labs, CA) which is a humanized anti-HLA-DR antibody; ABX-IL8 (Abgenix) which is a human anti-IL8 antibody; ICM3 (ICOS) which is a humanized anti-ICAM3 antibody; IDEC-114 (IDEC Pharmaceuticals/Mitsubishi) which is a primatized anti-CD80 antibody; IDEC-131 (IDEC Pharmaceuticals/Eisai) which is a humanized anti-CD40L antibody; IDEC-151 (IDEC Pharmaceuticals) which is a primatized anti-CD4 antibody; IDEC-152 (IDEC Pharmaceuticals/Seikagaku) which is a primatized anti-CD23 antibody; 5G1.1 (Alexion Pharmaceuticals) which is a humanized anti-complement factor 5 (C5) antibody; IDEC-151 (IDEC Pharmaceuticals/Smith-Kline Beecham) which is a primatized anti-CD4 IgG1 antibody; MDX-CD4 (Medarex/Eisai/Genmab) which is a human anti-CD4 IgG antibody; CDP571 (CellTech, UK) which is a humanized anti-TNF-α IgG4 antibody; LDP-02 (LeukoSite/Genentech) which is a humanized anti-α4β7 antibody; OrthoClone OKT4A (Ortho Biotech) which is a humanized anti-CD4 IgG antibody; ANTOVA™ (Biogen) which is a humanized anti-CD40L IgG antibody; TYSABRI® (Elan Pharmaceuticals) which is a humanized anti-VLA-4 IgG antibody; MDX-33 (Medarex/Centeon) which is a human anti-CD64 (FcγR) antibody; SCH55700 (Celltech, UK and Schering) which is a humanized anti-IL-5 IgG4 antibody; SB-240563 and SB-240683 (SmithKline Beecham) which are humanized anti-IL-5 and IL-4 antibodies, respectively; rhuMab-E25 (Genentech, CA) which is a humanized anti-IgE IgG1 antibody; IDEC-152 (IDEC Pharmaceuticals) which is a primatized anti-CD23 antibody; SIMULECT™ (Novartis Pharmaceuticals) which is a chimeric anti-CD25 IgG1 antibody; LDP-01 (Leukosite) which is a humanized anti-β2-integrin IgG antibody; CAT-152 (Cambridge Antibody Technology) which is a human anti-TGF-β2 antibody; and Corsevin M (Centocor) which is a chimeric anti-Factor VII antibody.

B. Effector Moieties for Conjugation

For the purpose of the present invention, the term “effector moiety” is used to refer to any molecular entity that may be linked to an antibody and thereby localize its effect to a specific biological target. The term “effector moiety” is intended to include labeling or reporting moieties (e.g. radioactive or fluorescent labels), as well as, therapeutic moieties including cytotoxic agents, chemotherapeutic agents, and immunostimulatory agents.

Thus, the effector moieties for labeling that may be conjugated to an antibody according the processes of the present invention include but are not limited to detectable labeling molecules for diagnosis and/or imaging including radio-opaque dyes, radio-contrast agents, fluorescent molecules, spin-labeled molecules, and enzymes.

In addition, the effector moieties that may be conjugated to an antibody according the processes of the present invention include but are not limited to therapeutic molecules including drugs, chemotherapeutic agents, cytotoxic agents, toxins, active fragments of toxins, or immunostimulatory agents.

“Cytotoxic agent” as used herein refers to substances that inhibit or prevent the function of cells and/or causes destruction of cells. Examples include radioactive isotopes (e.g., I¹³¹, I¹²⁵,Y⁹⁰, RE¹⁸⁶, Bi²¹², or other alpha- or beta- emitters), chemotherapeutic agents, and toxins such as enzymatically active toxins of bacterial, fungal, plant or animal origin, or fragments thereof. Suitable toxins and their corresponding fragments include diphtheria A chain, exotoxin A chain, ricin A chain, abrin A chain, curcin, crotin, phenomycin, enomycin, dolastatin 10, auristatins, such as auristatin E and auristatin F, calicheamicin, and the like (See, generally, “Chimeric Toxins,” Olsnes and Phil, Pharmac. Ther., 25, 355-381 (1982), and “Monoclonal Antibodies for Cancer Detection and Therapy,” eds. Baldwin and Byers, pp. 159-179, 224-266, Academic Press (1985), all of which are incorporated herein by reference.) In a preferred exemplary embodiment, the cytotoxic agents are dolastatin 10 and its synthetic analog, monomethyl auristatin E (MMAE), or maleimide-mono-methyl-valine-citrulline-auristatin-E (also referred to as “vcMMAE” or “Val-Cit-auristatin E”).

Examples of chemotherapeutic agents that may used as effector moieties with the anti-α5β1 antibodies of the present invention include, but are not limited to, adriamycin, doxorubicin, doxil, epirubicin, 5-fluorouracil, cytosine arabinoside (“Ara-C”), cyclophosphamide, thiotepa, busulfan, cytoxin, taxoids, e.g., paclitaxel (Taxol™, Bristol-Myers Squibb Oncology™, Princeton, N.J.), and doxetaxel (Taxotere™, Rhone-Poulenc Rorer), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), 5-FU, 6-thioguanine, 6-mercaptopurine, actinomycin D, VP-16, chlorambucil, melphalan, and other related nitrogen mustards. Also included in this definition are hormonal agents that act to regulate or inhibit hormone action on tumors such as tamoxifen and onapristone.

In addition, the related analogs and derivatives of these cytotoxic and chemotherapeutic agents may also used as effector moieties to produce antibody conjugates according to the present invention.

Alternatively, rather than use cytotoxic agents, immune stimulators that trigger T-lymphocytes and activate cytotoxic T lymphocytes (CTLs) may be conjugated as effector moieties according to method of the present invention. Examples of antibody conjugates with immune stimulator molecules include: (i) antibodies that are directed against a T-cell receptor or compounds that are able to bind to a T-cell receptor (see, e.g., EP 0 180 171 A1); (ii) compounds, such as antigens, mitogens, other foreign proteins, and peptides that activate cytotoxic T-cells (see e.g., EP 0 334 300 A1); (iii) MHC antigens, (see e.g., EP 0 352 761 A1); (iv) antigens against which the individual to be treated has immunity, (see e.g., WO 90/11779); and (v) an unnamed bacterial enterotoxin (see e.g., Ochi and Wake, UCLA Symposium: Cellular Immunity and the Immunotherapy of Cancers, Jan. 27-Feb. 3, 1990, Abstract CE 515, page 109).

C. Coupling Reactions Useful for Making Antibody Conjugates to Effector Moieties

An antibody conjugate is an antibody that is chemically coupled (i.e. linked, or bound) to an effector moiety. The two components may be coupled together by any of a variety of well-known chemical procedures for linking molecules to antibodies. For example, when the effector moiety is a protein and the second component is an intact immunoglobulin, the linkage may be by way of heterobifunctional cross-linkers, e.g., SPDP, carbodiimide, glutaraldehyde, or the like.

An effector moiety may be coupled (e.g., covalently bonded) to a suitable antibody either directly or indirectly (e.g., via a linker group). A direct coupling reaction between an effector moiety and an antibody may be utilized where each possesses a substituent (i.e., a chemical “handle”) capable of reacting with a substituent on the other.

Antibody substituents typically used for conjugation include:
(1) Carbohydrate structures. This structure may be oxidized to aldehyde groups that in turn are reacted with a compound containing the group H₂NNH— to the formation of a —C═NH—NH— group.
(2) Thiol group (HS—). The thiol group may be reacted with a compound containing a thiol-reactive group to the formation of a thioether group or disulfide group. Free thiol groups of proteins are present in cysteine residues and may be introduced onto proteins by thiolation or splitting of disulfides in native cysteine residues.
(3) Free amino groups (H₂N—) in amino acid residues. An amino groups may be reacted with a compound containing an electrophilic group, such as an activated carboxy group, to the formation of an amide group. The free amino group preferably is an amino terminal or the omega amino group of a lysine residue.
(4) Free carboxy groups in amino acid residues. A carboxy group may be transformed to a reactive (activated) carboxy group and then reacted with a compound containing an amino group to the formation of an amide group. However, precautions must then be taken to minimize amide formation with the amino groups that mostly are present together with carboxy groups in the same protein. The free carboxy group preferably is a carboxy terminal or a carboxy group of a diacidic alpha amino acid.

In one example, a nucleophilic group, such as an amino, or sulfhydryl group, on the antibody may be a substituent used in a coupling reaction with a carbonyl-containing substituent, such as an anhydride or an acid halide, or an alkyl substituent with a strong leaving group (e.g., a halide), on the effector moiety. In one particularly preferred coupling reaction of the present invention, sulfhydryl groups on the antibody undergo a facile coupling reaction with a maleimide substituent on the effector moiety (e.g., the cytotoxic agent, vcMMAE) to form a thio-ether linkage between the two components of the conjugate.

In some embodiments, it may be desirable to initially modify the antibody, or the effector moiety with a linker group before the coupling reaction. A linker group may be selected to increase the chemical reactivity of a substituent on an agent or an antibody, and thus increase the coupling efficiency. An increase in chemical reactivity may also facilitate the use of agents, or functional groups on agents, which otherwise would not be possible. Typically, the linker group is selected for its specific ability to undergo a facile coupling reaction with a particular substituent on the antibody (e.g., a linker with a terminal maleimide or succinimide group). However, in some embodiments the linker may also be selected to function as a spacer that distances the antibody from the effector moiety. Such spacing may be necessary in order to maintain the biological effect of the moiety (e.g., avoid antibody interference with its binding capabilities).

It will be evident to those skilled in the art that a variety of bifunctional or polyfunctional reagents, both homo- and hetero-functional (such as those described in the catalog of the Pierce Chemical Co., Rockford, Ill.) , may be employed as the linker group. Coupling may be affected, for example, through amino groups, carboxyl groups, sulthydryl groups of oxidized carbohydrate residues. There are numerous references describing such methodology, e.g., U.S. Pat. No. 4,671,958, which is hereby incorporated by reference herein.

Where a effector moiety, such as a cytotoxic agent, is more potent when free from the antibody portion of the conjugates of the present invention, it may be desirable to use a linker group which is cleavable during or upon internalization into a cell. A number of different cleavable linker groups have been described, such as disulfide bond linkers, hydrazone bond linkers, and dipeptide bond linkers, etc. The mechanisms for the intracellular release of an agent from these linker groups include, but are not limited to, cleavage by reduction of a disulfide bond (e.g., U.S. Pat. No. 4,489,710, which is hereby incorporated by reference herein), by irradiation of a photolabile bond (e.g., U.S. Pat. No. 4,625,014, which is hereby incorporated by reference herein), by hydrolysis of derivatized amino acid side chains (e.g., U.S. Pat. No. 4,638,045, which is hereby incorporated by reference herein), by serum complement-mediated hydrolysis (e.g., U.S. Pat. No. 4,671,958, which is hereby incorporated by reference herein), and acid-catalyzed hydrolysis (e.g., U.S. Pat. No. 4,569,789, which is hereby incorporated by reference herein).

D. Therapeutic Utility of Antibody Conjugates

As mentioned above, antibody conjugates of effector moieties comprising cytotoxic or chemotherapeutic agents have been developed for a wide range of disease indications, most significantly, cancers (e.g. breast, prostate, ovarian, colon, lung cancers), autoimmune diseases (e.g., systemic lupus erythematosus (SLE), multiple sclerosis, rheumatoid arthritis, psoriasis, Crohn's disease, ulcerative colitis), infectious bacterial diseases (e.g., disease states caused by the bacteria Streptococcus pneumoniae, Neisseria gonorrheae, or Staphylococcus aureus) and viral diseases (e.g., herpes, hepatitis A, B and C). Specific types of cancer, autoimmune and infectious disease that may be targeted using an antibody conjugate are well-known in the art. For example, conjugates of the chemotherapeutic, doxorubicin, and antibodies to the cancer antigens, BR96 and BR64, have been extensively developed (See, e.g., Trail et al., “Cure of Xenografted Human Carcinomas by BR96-Doxorubicin Immunoconjugates” Science 1993, 261, 212-215; Trail et al., “Effect of Linker Variation on the Stability, Potency, and Efficacy of Carcinoma-reactive BR64-Doxorubicin Immunoconjugates” Cancer Research 1997, 57, 100-105). In addition, many other antibodies that target tumor associated antigens and are known to be internalized have been conjugated, or proposed as candidates for conjugation, with chemotherapeutic effector moieties (see, e.g., Meyer, D. L., et al., “Recent advances in antibody-drug conjugates for cancer therapy,” Annual Reports in Medicinal Chemistry 2003, 38:229-237; Franke, et al., “Cell surface receptor-targeted therapy of acute myeloid leukemia: a review” Cancer Biother. Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998). Because of their general applicability for the efficient, high yield production of antibody conjugates with cytotoxic agents, the methods of the present may be employed in targeting these disease indications.

Other non-limiting examples of specific cancer antigens that may be targeted with an antibody conjugate include: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), MUC1 (carcinomas), placental alkaline phosphatase (carcinomas), prostate specific antigen (prostate), prostatic acid phosphatase (prostate), epidermal growth factor (carcinomas), MAGE-1 and MAGE 3 (carcinomas), anti-transferrin receptor (carcinomas), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), and CD40 (lymphoma).

Additional specific antigens that may be targeted with antibody conjugates of the present invention include α5β1 integrin, LIV-1, TMEFF2, ESSL (E-selectin, endothelial adhesion molecule 1), GPR39, Delta-like 3 protein, TIA-2 lung type-I CMAG, ATP-binding cassette, hLIb, G protein-coupled receptor 64 (GPR64), solute carrier family 30, G-protein-coupled receptor 49, FLJ32082, Hepatitis A virus cellular receptor 1, APK1B, and SLC15A2 solute cattier family 15. A conjugate of the anti-TMEFF2 antibody, Pr1 and a derivative of the cytotoxic agent auristatin-E (vcMMAE) has been prepared and shown to be effective in shrinking in vivo xenograft models of prostate cancer tumors (see, e.g, U.S. patent Publication Ser. No. 2004/0096392 A1, which is hereby incorporated by reference herein). In another preferred example, an antibody against GPR64 (a validated ovarian cancer target also referred to as Ov1, or OAM6) has also been conjugated to vcMMAE. This antibody conjugate has shown to effectively kill cancer cells (H460) in vitro (see, e.g, U.S. patent Publication Ser. No. 2004/0197325 A1, which is hereby incorporated by reference herein).

III. An Alcohol Solvated Antibody Conjugation Reaction

In one embodiment, the present invention is directed to a process for producing a conjugate of an antibody and at least one effector moiety, said process comprising: performing a reaction coupling the antibody to the effector moiety in a solution comprising an alcohol. The process may be carried out wherein the alcohol concentration of the coupling reaction solution is a concentration of about 5% to 50% by volume. The specific alcohol concentration may be selected based on known properties of the desired antibody and/or effector moiety to be conjugated, and/or the specific alcohol selected. In one embodiment, pre-screening may be carried out wherein the alcohol concentration is determined which allows the highest antibody concentration may be maintained with little or no aggregation during the conjugation process. Such pre-screening is routine and well-known to one of ordinary skill in the art of antibody purification and processing. In a preferred embodiment of the present invention, the alcohol concentration that minimizes antibody aggregation and allows high concentration is preferably about 10-50% alcohol by volume. Preferred conjugation reaction alcohol concentrations therefore include at least about 5%, 10%, 20%, 30%, or 50% alcohol by volume, and the various concentration ranges between 5% and 50% alcohol by volume.

For the purpose of the present invention, the term “alcohol” is used with its common ordinary meaning, that is a class of alkyl compounds containing a hydroxyl group. According to the processes of the present invention, the preferred alcohols include straight and branched chain aliphatic alcohols with six or fewer carbons, including, but are not limited to, methyl alcohol, ethyl alcohol, n-propyl alcohol, isopropyl alcohol, n-butyl alcohol, sec-butyl alcohol, tert-butyl alcohol, n-pentyl alcohol, neopentyl alcohol, n-hexyl alcohol. In a particularly preferred embodiment, the alcohol is ethanol or isopropyl alcohol. The structural and solvent characteristics of these simple alcohols are well-established. Moreover, they may be obtained relatively inexpensively in highly purified liquid form well-suited for use in large-scale commercial processing of biologicals. Although in a preferred embodiment, a single alcohol is used during the conjugation reaction, the present invention also includes wherein combinations of alcohols are used as a solvent. For example, it may be determined that the increased solubility for a particular antibody and/or effector compound is achieved in a solution comprising 50% by volume of a 50:50 mixture of ethanol and isopropanol. Information on the use of solvent combinations may be found in chemical literature and is well known to those of ordinary skill in art.

Importantly, the alcohol solvated conjugation reaction of the present invention may be carried out with antibody concentrations higher than those achieved in prior art reactions without concomitant antibody aggregation resulting in a failed or low yielding process. For example, the process for producing a conjugate of an antibody and at least one effector moiety according to the present invention may be performed wherein the antibody concentration is at least 1, 5, 8, 10, 12, 15, 18, 20, 25, 30, or 50 mg/mL, with a preferred range of 10 mg/mL-50 mg/mL. Exemplary antibody concentrations useful in the methods disclosed herein include 10, 15, 20 and 25 mg/mL.

Another result of the low aggregation achieved with the alcohol-based conjugation reaction of the present invention, are relatively high percentages of antibody conjugate monomer species. Thus, the methods disclosed herein result in the formation of antibody conjugates with more than about 50%, 80%, 85%, 90%, 95%, 99%, or even higher percentages of monomer conjugate species. In addition, the alcohol-solvated conjugation reaction of the present invention results in antibody conjugates wherein multiple effector moieties are coupled to each antibody molecule (e.g. at each available reduced thiol group on the antibody). In preferred embodiments, the resulting effector moiety to antibody ratio is at least 1, 2, 3, 4, 5, 6, 7, 8, 10, 15, 20, preferably, in a range of 5-15, and more preferably, in a range of 6-10.

Another significant feature of the presently disclosed alcohol-solvated antibody conjugation method is that it may be carried out at temperatures above 4° C., including the exemplary temperatures of about 10° C., 15° C., 20° C., and 25° C., and preferably at about room temperature (i.e. about 22° C.). Consequently, the present invention includes a method wherein the antibody conjugation reaction is carried out at room temperature, and without any refrigeration.

In another aspect, the alcohol-solvated antibody conjugated method of the present invention is particularly well-suited for scale-up to large quantities of antibody conjugate production. In preferred embodiments, the method may be employed to produce more than about 10, 30, 50, 100, 150, 200, 300, 400, 500, 1000, 2000, or 5000 mg antibody conjugate in a single batch conjugation reaction.

IV. A Single-Pot Process for Making Antibody Conjugates to Effector Moieties

As described above the alcohol solvated antibody conjugation reaction has numerous advantages that provide utility across a broad range of antibody conjugations. In one preferred embodiment, the alcohol solvated reaction may be employed in a method for making antibody conjugates comprising: (a) exposing an antibody to a reducing agent; (b) performing a reaction coupling the reduced antibody to an effector moiety in a solution comprising an alcohol; and (c) separating the antibody conjugate from uncoupled effector moiety. As described further below, in a particularly preferred embodiment, the present invention includes a method wherein all three steps are conducted in a “single-pot” system comprising a recirculation apparatus and an tangential flow filtration (TFF) cell, wherein the initial antibody, reduced antibody, and/or antibody conjugate product are not removed from the system until all three steps of the method are complete. In the single-pot embodiment, TFF is used for buffer exchange through diafiltration and separation of unreacted effector molecules, thereby foregoing steps involving desalting columns that require removal of the antibody from the system.

In step (a), at least one disulfide-bond between cysteine residues in an antibody may be reduced by a reducing agent so as to expose the reactive sulflhydryl groups. The exposed sulfhydryl groups create a facile chemical handle for generating a thio-ether linkage to connect one or more effector moieties to the antibody. A number of reducing agents for this purpose are well-known in the art and commercially available, such as dithiothreitol (DTT) or β-mercaptoethanol. In general, the selected reducing agent is added to a solution containing the antibody at a concentration of at least about 1, 2, 3, 4, 5, 7, 10, 15, 20, 30, 50, or 100 mM. The reduction process is then conducted for a period of time until completion is established by monitoring the antibody's reduction and stability using the methods known in the art. In an exemplary embodiment, the reduction process is carried out at an elevated temperature, such as 37° C., for about 10 to 50 minutes, preferably, for about 30 minutes.

For the purpose of the present invention, the residual reducing agent (or the residue of the reducing agent) refers to reducing agent that did not react, i.e. the reducing agent remaining in the antibody mixture after the reduction reaction is complete. In a preferred embodiment, after the reduction reaction is deemed complete, the residual reducing agent is removed and buffer is exchanged via diafiltration in the TFF system and the reduced state of the molecule is stabilized by the addition of a chelating agent, preferably diethylenetriaminepentaacetic acid (DTPA). Typically, the TFF system is set up with a flat plate ultrafiltration membrane cassette (or “ultrafiltration skid”), although alternative ultrafiltration membrane configurations may be employed. An ultrafiltration membrane may be selected having a molecular weight cutoff ranging from 5 kD to 500 kD, form 10 kD to 200 kD, preferably, from 30 kD to 50 kD, depending on the molecular weight of the antibody. Saline solutions, such as PBS, are usually used to diafilter the residue reducing agents. After the reducing reaction, the reaction mix comprising the reduced antibodies and the residue reducing agents are buffer exchanged with the saline solution with buffer exchange volume of about 1-50, preferably 5-15, and more preferably, about 10. Antibodies are diafiltered into the saline solution while the unwanted residual reducing agent is eluted with the old buffer. In order to prevent the oxidation of the reduced sulfhydryl groups of the eluted antibody, a chelating agent should be added into the saline solution. The chelating agents are commonly used and known in the art, including, but not limited to EDTA, and DPTA.

In step (b), the selected alcohol, for example, isopropyl alcohol, is added to the buffer comprising the reduced antibody. The volume of alcohol added should result in a final concentration by volume of at least about 5%, 10%, 20%, 30%, or 50%, preferably at about 10% to 50%, and more preferably at about 20% alcohol. In some embodiments, the reduced antibody solution may be diluted to a final concentration of at least about 1, 2, 3, 5, 10, 15, 20, or 50 mg/ml before the addition of the alcohol solution.

Also in step (b), the solution comprising the effector moiety to be conjugated (e.g., vcMMAE) is added to the alcohol solution comprising the reduced antibody. Typically, the effector moiety is added at a molar ratio 2-fold, 4-fold, 6-fold, 8-fold, or 10-fold in excess of the number of moles of antibody, preferably 10-fold.. Generally, the preferred ratio corresponds to a slight excess relative to the number of available sulfhydryls on the reduced antibody. Once the effector moiety is added, the reaction solution is stirred well and incubated at room temperature (i.e., about 20-25° C.) for about 10-60 minutes, preferably, for about 30 minutes.

After the conjugation reaction of step (b) is completed, in step (c) the residual effector moiety (e.g. unreacted vcMMAE) is removed by separated and removed, preferably by diafiltration into the desired formulation buffer (e.g., PBS) via the same approach used to remove residual reducing agent in step (a). The resulting formulated antibody conjugate is formulated in buffer at a concentration of at least about 5-100 mg/mL, preferably 10-50 mg/mL.

The antibody conjugates resulting from this three step process embodiment, may then be characterized for concentration, effector moiety substitution (i.e., effector moiety/antibody ratio), percentage of monomer, specificity, and potency, according to methods well known in the art. For example, the concentration of the antibody conjugates may be determined using UV/VIS spectrophotometry. Typically, the antibody conjugates should have an A₂₈₀/A₂₆₀ ratio of 0.25 to 2.00, and preferably, 0.85 to 1.15. The effector moiety substitution ratio (e.g. the number of cytotoxic agents per antibody) may be evaluated by approaches known in the art, for example, MALDI-TOF mass spectrometry (see more details in Example 1C). The percentage of monomer (or alternatively amount of aggregates) among the produced antibody conjugates may be measured by size-exclusion HPLC. The binding specificity of the antibody conjugates may be analyzed by methods known in the art of immunology, such as ELISA or Biacore assays. The effect of the conjugate must be evaluated according to the type of effector moiety. For example, the potency of a cytotoxic agent targeted by an antibody to a cancer antigen may be evaluated by administering the conjugates to cancer cells in vitro (for example, cancer cell lines cultivated in cell cultures) and/or in vivo (in cancer mouse model) and monitoring the amount of induced cell death by the conjugates compared to the control.

In a preferred embodiment, the entire process of making antibody conjugates, comprising steps a, b, and c, described above is carried out in an “single pot” system (i.e. wherein a single solution of antibody, reduced antibody, and/or antibody conjugate remains in the system until all three steps are complete). The single-pot system may comprise any reaction/separation system well-know for such purposes. For example, the single-pot system may comprise an ultrafiltration skid, or a recirculation pump connected to a TFF apparatus (e.g. an ultrafiltration cassette system). The single-pot system may be considered “enclosed” in that the antibody solution (i.e. the initial starting antibody, the reduced antibody, and the antibody conjugate product) is never transferred out of the system during the course of the three step process yielding the antibody conjugate product. The system is not fully enclosed however, with respect to the reactants (i.e. reductant, and effector moiety), alcohol and buffer, which are fed into the system, and removed as waste after reaction, during the course of the process.

In one aspect, the enclosed system is an ultrafiltration skid. After the reduction reaction, the reaction mixtures comprising the reduced antibodies and the residual reducing agents are added to the ultrafiltration skid, wherein the residual reducing agents are washed away via diafiltration while the reduced antibodies retain in the skid. The alcohol and the cytotoxic agents are then added to the skid for the conjugation process. After the conjugation, the residual cytotoxic agents are filtrated out via diafiltraton again, while the produced antibody conjugates stays in the skid. Alternatively, the reduction reaction takes places in the skid. The antibody and reducing agent are mixed in the skid for the optimal period of time for the reduction reaction to be completed. Then, the residual reducing agents are washed away via diafiltration. In some embodiments, the reduction reaction is performed at a temperature higher than the room temperature, such as at 37° C. The antibody and the reducing agent can be transferred to the skid and warmed up to 37° C. The reducing agent (such as, DTT) is then added and mixed with the antibody. After the reduction is completed, the skid temperature is reduced to the room temperature before the diafiltration of the residual reducing agents.

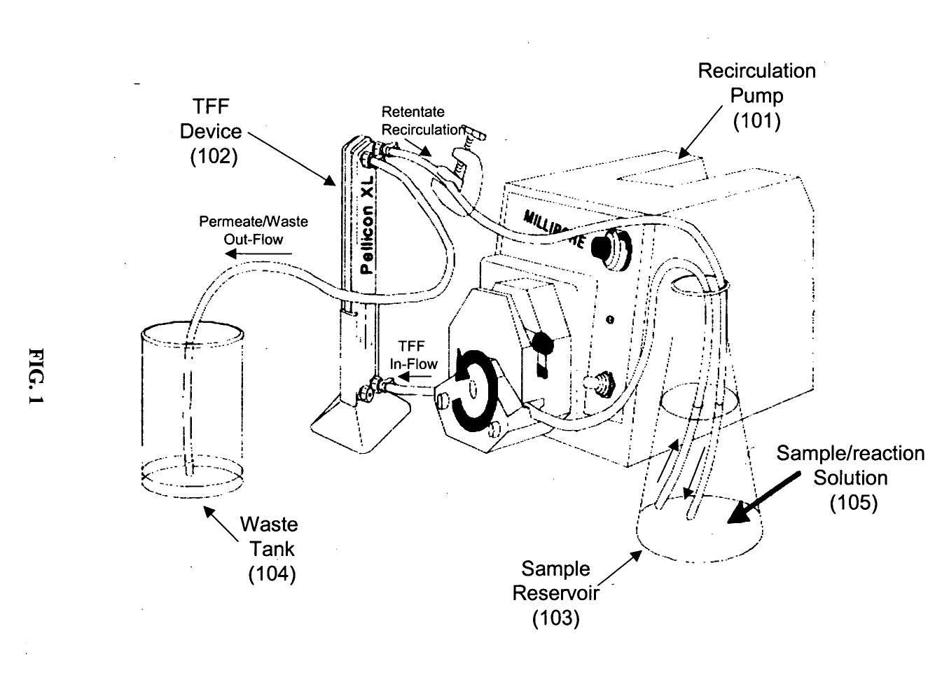

In another aspect, the antibodies undergo conjugation while remaining enclosed in a recirculating system comprising a tangential flow filtration (TFF) system (e.g. a Millipore Pellicon XL ultrafiltration skid/cassette with a Biomax 30K 50 cm² membrane), a recirculation pump, sample reservoir vessel (e.g., glass flask), and a waste tank. As shown in FIG. 1, the recirculation pump (101) is connected through tubing to the TFF device (102) comprising an ultrafiltration membrane, which is connected through tubing to both the waste tank (104) and the sample reservoir (103). Typically, starting antibodies and the reducing agent are added to the recirculation tank in the beginning of the production process. The reduction reaction will then take place in the sample reservoir for the optimal period of time. When the reaction is completed, the reduced antibodies are retained inside the recirculation system while the residual reducing agents are selectively removed by molecular sieving and diafiltration via the tangential flow filtration device, and passed into the waste tank where they may removed through an opening in the waste tank. The selected alcohol (for example, isopropyl alcohol) is then added to the reduced antibodies in the ultrafiltration cassette. The conjugation reaction will be carried out in the solution containing the alcohol. The residual cytotoxic agents are selectively, by molecular sieving with tangential flow filtration, passed through the filter into the waste tank via diafiltration. The end products (such as the formed antibody conjugates) are transported into the sample tank and collected through an opening thereof.

The above-described recirculating system may be easily adapted to a wide range of commercially available recirculating pumps, TFF devices, and reaction vessels, beyond those embodied herein. Exemplary equipment may be obtained from bio-processing pump and filtration equipment companies, such as Millipore (Billerica, Mass.).

For the purpose of the present invention, the residual cytotoxic agents (or the residues of the cytotoxic agents are cytotoxic agents that are not conjugated to the antibodies after the conjugation process.

The following examples illustrate specific embodiments and are not intended to limit the scope of the inventions disclosed herein.

### EXAMPLES

### Example 1

Preparation and Characterization of a vcMMAE Conjugate of the Pr1 Antibody

Overview

A high-yield, lab-bench scale process was used to prepare a conjugate of vcMMAE to the anti-TMEFF2 antibody, Pr1. The preparation and characterization of the Pr1 antibody has been previously disclosed in U.S. patent Publication Ser. No. 2004/0096392 A1, which is hereby incorporated by reference herein. The preparation and characterization of vcMMAE has been previously disclosed in Doronina, et al., Nature Biotechnology 21: 778-784 (2003). The high quality of the Pr1-vcMMAE conjugate prepared, as described below, was further characterized using spectrophotometry and MALDI-TOF mass spectrometry.

A. Conjugate Preparation Protocol
1) Pr1 antibody was provided at ˜20 mg/mL concentration in pH 7.4, PBS. Pr1 concentration was determined spectrophotometrically based on A₂₈₀ in a 1 cm cuvette using the relation, A₂₈₀/1.4=Ab concentration (mg/mL). In addition, the starting Pr1 antibody solution was characterized as greater than 99% monomer species by HPLC sizing using a TOSOH Biosep TSK-G3000SWXL sizing column and Bio-Rad #151-1901 molecular weight standards.
2) A 1 M DTT/water solution was prepared (150 mg of DTT dissolved in 1.0 ml water). This solution was used within 15 minutes in order to maintain adequate DTT activity.
3) Reduced Pr1 antibody was produced by adding an aliquot of 1 M DTT solution to the starting Ab solution so that the resulting concentration was 10 mM DTT. This Ab+DTT reaction solution was incubated and rotated at 37° C. for 30 minutes.
4) The above reduced Ab solution was loaded into a Millipore Pellicon XL 50 tangential flow filtration (TFF) apparatus (Biomax 30K 50 cm² membrane, Masterflex pump, 12 ml dead volume), that was previously equilibrated with at least 200 ml of PBS/DPTA buffer (PBS+1 mM DTPA, pH 6.2). The solution Ab+DTT solution was buffer exchanged into PBS/DTPA until the filtrate shows no residual DTT in a 5,5′-dithiobis(2-nitrobenzoic acid) (DTNB) assay (DTNB solution: 40 mg in 1 mL MeOH+9 mL of 100 mM NaPO₄) (see, e.g., Riddles, P. W., et al., “Ellman's reagent: 5,5′-dithiobis(2-nitrobenzoic acid)-a reexamination” Anal. Biochem. 94: 75-81(1979)).
5). The resulting reduced Ab solution was diluted with PBS/DTPA to a concentration of 10 mg/ml. The concentration of thiols was determined spectrophotometrically using the relation, (A₄₁₂/14.15)/0.9=mmoles thiol. Ab concentration was determined spectrophotmetrically using the relation, (A₂₈₀/1.4)/150=mmoles Ab. The resulting yield of thiols per Ab was determined to be 8±2.
6) To the above reduced Ab solution was added, with vortexing, a volume of isopropyl alcohol (EPA) corresponding to 20% of the starting volume (e.g. 10 mL IPA added to 50 mL reduced Ab solution).
7) A 10 mM solution of vcMMAE in DMSO was prepared. A volume of the vc-MMAE solution corresponding to a 10:1 molar excess of vcMMAE to Ab was added to the reduced Ab solution quickly with vortexing. This reaction mixture was stirred at room temperature for 30 minutes.
8) The reaction mix was exchanged into PBS, pH 7.4 using the TFF unit. Add PBS was added to adjust the final Ab conjugate concentration to 10 mg/mL.
9). After final recovery from the TFF unit, the formulated conjugate was run through a 0.2 μM filter into a sterile container
10). Final yield (i.e. total mg) of Pr1-vcMMAE conjugate was determined spectrophotometrically (HP-8453 Spectrophotometer).

Results: Pr1-vcMMAE Conjugate Yield

Starting with 428 mg Pr1 Ab, a total of 400 mg of Pr1-vc-MMAE conjugate was recovered from a single batch preparation according to the protocol described above.

B. HPLC Determination of Percentage Monomer of Pr1-vc-MMAE Conjugate Methods
1). The HPLC (TOSOH Biosep TSK-G3000SWXL) sizing column was run at 1.5 ml/min for an isocratic 10 min run with PBS as the mobile phase.
2). 10 μl of MW standards (Bio-Rad #151-1901) were injected and relative retention time of IgG was determined.
3). 25 μl samples of the starting Pr1 Ab and the final Pr1-vcMMAE conjugation product.
4). The HPLC software was use to integrate the peak(s) and non-monomer shoulder.

Results: Percentage Monomer

The HPLC sizing results indicated that the Pr1-vcMMAE conjugate was more than 99% monomer.

C. Determination vc-MMAE Substitution Number with MALDI-TOF Mass Spectrometry Methods
1). A saturated solution of sinipinic acid (SA) in 70% acetonitrile/30% water was prepared.
2). Two samples were prepared corresponding to the Pr1 alone and the Pr1-vcMMAE conjugate preparation. The samples were prepared by adding 1 μl of either the Ab alone or the conjugate solution (at 1 mg/ml concentration) to 10 μl of the SA solution with vortexing.
3). 1 μl of each sample was deposited onto the MALDI-TOF sample plate at 3 different spots. The spots were allowed to dry before loading the sample plate into the mass spectrometer.
4). Six measurements were recorded for each of the samples. Data was acquired using the IgG full spectrum method.
5). The number of vcMMAE molecules per Pr1 was calculated by subtracting the mass of the starting Pr1 antibody sample from the total mass of the Pr1-vcMMAE conjugate sample. This difference was then divided by 1316 to obtain the number of vcMMAE molecules per antibody.

Results

The drug substitution ratio of the produced Pr1-vcMMAE conjugates was tested by mass spectrometry by following the protocol described above. The results indicated that the conjugates so produced had on average 7.8 vcMMAE molecules per antibody.

### Example 2

Preparation of the Pr1-vc-MMAE Conjugate in a Recirculation Apparatus

The high-yield process for the conjugation of Pr1 with vcMMAE described above may be carried out in a recirculation apparatus according to the method below. The use of recirculation apparatus creates a “single pot” closed system minimizing the risk of exposure to the cytotoxic agent and thereby resulting in greatly enhanced process safety.
1). The recirculation apparatus (Millipore Corporation, Billerica, Mass.) is assembled as shown in FIG. 1. A sample tank (also called a recirculation tank) is connected to a pump, which is then connected to an ultrafiltration skid (Pellicon XL). The ultrafiltration skid is connected to a waste tank.
2). 400 mg Pr1 antibody at 20 mg/mL concentration is added to the recirculation tank. A volume of DTT in a 1M stock solution is added to the recirculation tank so that the circulating DTT concentration is 10 mM. The Pr1 antibody and DTT are allowed to incubate at 37° C. for about 30 minutes. The DTT results in reactive sulfhydryl groups on the Pr1 thereby enabling the use of common thio-ether conjugation chemistry to attach the desired drug molecule, in this case, vcMMAE.
3). The Pr1/DTT reaction mix then is pumped to a 30K or 50K molecular weight cut-off ultrafiltration skid and buffer exchanged by diafiltration into PBS/DPTA (pH 6.2) at room temperature (20-25° C.). The DPTA chelating agent prevents oxidation of the reduced sulfhydryl groups on the Pr1 antibody. The reduced antibody is retained inside the ultrafiltration skid and becomes retentate, while the residual DTT is washed into the waste tank and collected. The residual DTT concentration can be monitored via DTNB assay, or other assay for quantitation of free thiol.
4). Once the residual DTT is washed away, the filtrate valve between the waste tank and the ultrafiltration skid is closed. The reduced Pr1 is then diluted ˜2-fold to 10 mg/ml and isopropyl alcohol (IPA) is added to the recirculation tank to a final concentration of 20% (v/v).
5). 10 mM vcMMAE stock solution then is added into the reduced Pr1 (in 20% IPA) until a final 8% concentration (w/w) vcMMAE:Pr1 is achieved. This reaction mix then is recirculated in the system at room temperature for 30 minutes.
6). The filtrate valve is then opened and the reaction mixture in the recirculation tank is pumped into the ultrafiltration skid. The residual vcMMAE is diafiltered out into the waste tank using formulation buffer (PBS at pH 7.4) and the Pr1-vcMMAE is retained in the skid and exchanged into the PBS, pH 7.4 buffer.

Results

The entire Pr1-vc-MMAE conjugation process, including the DTT reduction, the vc-MMAE conjugation reaction, the residual cytotoxic agent removal, and the final antibody conjugate product formulation, is performed at high concentration in a single recirculating ultrafiltration system. Each step is conducted and completed in its entirety by recirculating and mixing the reaction reagents within the recirculation system, and a single tank captures all of the waste.

### Example 3

Inhibition of in Vivo Tumor Growth by Pr1-vcMMAE

To examine the effects of Pr1-vcMMAE in vivo, tumor xenografts of LNCaP (human prostate cancer cells) were grown in immunocompromised male SCID mice and were allowed to reach 50-100 mm³ in size. Mice were then randomized into groups and were treated with Pr1-vcMMAE or a control vcMMAE-conjugated antibody.

A. Generation of SCID Mice with LNCaP or CWR22 Xenografts

Immunocompromised mice CB-17 SCID (strain C.B-Igh1/IcrTac-Prkdc) were purchased from Taconic Farms, (Germantown, N.Y.). Studies were initiated using male mice between the ages of 6-12 weeks (˜20 grams in weight).

For LNCaP (FGC) implantations, 1×10⁷ cells in 50:50 volume of Iscove's media:matrigel were inoculated subcutaneously on the right flank of animals. For CWR22 tumor fragments were implanted subcutaneously on the right flank of animals. Tumors were allowed to establish until reaching an average of 50-100 mm³ as determined by caliper measurement and calculated by p/6×length×width×height. Animals were then randomized into groups for each of the desired treatment arms. The Pr1-vcMMAE antibody drug conjugate (ADC) was delivered either intraperitoneally or intravenously every 4 days (for 6-12 doses as indicated). Dosing was based on the calculation of drug equivalent (using auristatin E mol wt of 708) of each ADC. The concentration corresponding to the complete ADC (antibody+auristatin E+linker) is indicated and was an approximate concentration dependent on the number of drugs conjugated (usually 6-8) per antibody-for that particular ADC preparation.

Tumor volume was measured twice weekly and clinical and mortality observations were performed daily according to IACUC regulations.

Results

The results showed that Pr1-vcMMAE (0.215 mg/kg auristatin E, i.e. ˜5 mg/kg of ADC) delivered either intraperitoneally or intravenously to LNCaP-bearing mice resulted in significant inhibition of tumor growth. The anti-tumor effect of Pr1-vcMMAE correlated well with a decrease in serum prostate-specific antigen (PSA) levels, a serum marker often used as a surrogate for prostate cancer tumor burden. Pr1-vcMMAE treated mice exhibited serum PSA levels of less than 10 ng/ml at the end of the study, while control-vcMMAE treated mice had PSA levels well above 100 ng/ml.

The Pr1-vcMMAE was well tolerated as assessed by body weight. In contrast both groups receiving control ADC in which LNCaP tumors continued to grow had to be removed from study at day 54 post-implantation due to weight loss exceeding 20% (as per IACUC regulations). We have found that mice bearing LNCaP tumors are particularly susceptible to weight loss and that tumor-burdened mice often have to be sacrificed while the tumors are still relatively small (400-700 mm³). Mice treated with Pr1-vcMMAE at first exhibited a slight decrease in weight, which was attributed to tumor burden. However, after several doses of Pr1-vcMMAE, the weights of the animals stabilized and actually increased, indicating that Pr1-vcMMAE is well tolerated and is protective against weight loss due to tumor burden.

Although the invention has been described with reference to the presently preferred embodiments, it should be understood that various modifications may be made without departing from the spirit of the invention.

All publications, patents, and patent applications are herein incorporated by reference in their entirety to the same extent as if each individual patent, patent application, or web site was specifically and individually indicated to be incorporated by reference in its entirety.

1. A process of producing a conjugate of an antibody and at least one effector moiety, said process comprising: performing a reaction coupling the antibody to the effector moiety in a solution comprising at least about 5% by volume of an alcohol.

2. The process according to claim 1, wherein said alcohol is selected from the group consisting of ethanol and isopropyl alcohol.

3. The process according to claim 1, wherein said solution comprises alcohol at a concentration of about 10% to 50% by volume.

4. The process according to claim 1, wherein said antibody in the solution has a concentration of about 10 mg/mL to 20 mg/mL .

5. The process according to claim 1, wherein the amount of antibody coupled in the reaction is about 100 mg to 400 mg.

6. The process according to claim 1, wherein said reaction is performed at about 20° C. to 25° C.

7. The process according to claim 1, wherein said antibody conjugate comprises a monoclonal antibody.

8. The process according to claim 1, wherein said antibody conjugate comprises a chimeric, humanized, or fully human antibody.

9. The process according to claim 1, wherein said effector moiety is selected from the group consisting of a therapeutic moiety and a reporting moiety.

10. The process according to claim 1, wherein said effector moiety is a therapeutic moiety selected from the group consisting of diphtheria A chain, exotoxin A chain, ricin A chain, abrin A chain, calicheamicin, curcin, crotin, phenomycin, enomycin, dolastatin 10, auristatin E, auristatin F, and vc-MMAE.

11. The process according to claim 1, wherein said antibody is Pr1 and said effector moiety is vc-MMAE.

12. A method of producing an antibody conjugate with at least one effector moiety, said method comprising:
a. contacting an antibody to a reducing agent;
b. performing a reaction coupling the reduced antibody to an effector moiety in a solution comprising at least about 5% by volume of an alcohol; and
c. separating the antibody conjugate from uncoupled effector moiety.

13. The method according to claim 12, wherein said reducing agent is dithiothreitol.

14. The method according to claim 12, further comprising removing residual reducing agent by ultrafiltration between step a and step b.

15. The method according to claim 12, wherein said uncoupled effector moiety is removed by diafiltration.

16. The method according to claim 12, wherein all three steps are performed in a single-pot system comprising a single solution of antibody, reduced antibody, and/or antibody conjugate that remains in the system until all three steps are complete.

17. The method according to claim 12, wherein each step of said method is performed in an ultrafiltration skid.

18. The method according to claim 12, wherein each step of said method is performed in a recirculating system comprising a recirculation pump connected to a tangential flow filtration device.

19. The method according to claim 18, wherein said antibody is Pr1 and said effector moiety is vc-MMAE.
